(19) United States
(12) Patent Application Publication
(10) Pub. No.: US 2021/0035666 A1
(43) Pub. Date: Feb. 4, 2021

(54) INTEGRATING DISTRIBUTED SYSTEMS USING BIOMETRIC IDENTIFICATION

(71) Applicant: Alclear, LLC, New York, NY (US)

(72) Inventor: Joe Trelin, New York, NY (US)

(21) Appl. No.: 16/934,325

(22) Filed: Jul. 21, 2020

Related U.S. Application Data
(60) Provisional application No. 62/879,630, filed on Jul. 29, 2019.

(51) Int. Cl. G16H 10/60; G06F 16/174; G06F 21/32

(52) U.S. Cl. CPC G16H 10/60; G06F 21/32; G06F 16/1748

(57) ABSTRACT

A system uses a digital representation of a biometric to match and/or dedupe first and second records respectively stored in first and second systems. In various examples, an identification system may be used to determine an identity using a digital representation of a biometric, locate a first record in the first system and a second record in a second system, and dedupe the first and second records. In a number of examples, an identification system may be used to locate a first record in a first system and a second record in a second system using identity information, determine that the first record and second record cannot be verified as associated with a same person within a threshold certainty, and determine whether a person associated with the first record is associated with the second record using a digital representation of a biometric for the person.

### CROSS-REFERENCE TO RELATED APPLICATION(S)

This application is a nonprovisional patent application of and claims the benefit of U.S. Provisional Patent Application No. 62/879,630, filed Jul. 29, 2019 and titled “Integrating Distributed Systems Using Biometric Identification,” the disclosure of which is hereby incorporated herein by reference in its entirety.

### FIELD

The described embodiments relate generally to biometric identification. More particularly, the present embodiments relate to integrating distributed systems using biometric identification.

### BACKGROUND

An increasing number of systems electronically store personal records about people. For example, a variety of different hospital and/or other medical provider systems store various medical records about people. By way of another example, a variety of different banks and/or other financial systems store various financial records about people.

Additionally, use of biometrics (such as one or more fingerprints, blood vessel scans, palm-vein scans, voiceprints, facial images, retina images, iris images, deoxyribonucleic acid sequences, heart rhythms, gaits, and so on) to establish a person's identity is increasingly common. Biometrics may be used to unlock electronic devices, retrieve sensitive information, enter secure areas (such as airport terminals, event venues, and so on), rent or purchase goods and/or services, and so on. A variety of different technologies may be used in a variety of different implementations to establish a person's identity using biometrics.

Some systems may use biometrics in association with records stored about people. For example, the records may store biometric data about people. By way of another example, biometric identification may be used to control access to records about people.

### SUMMARY

The present disclosure relates to integrating distributed systems. One or more digital representations of biometrics may be used to match and/or dedupe first and second records respectively stored in first and second systems. Deduping may involve removing duplicate information from one or more of the records and/or the records themselves, linking the records, copying information from one record to the other, and so on. In various examples, an identification system may be used to determine an identity for a first system using a digital representation of a biometric, locate a first record in the first system associated with the identity using the digital representation of the biometric, locate a second record in a second system associated with the identity using the digital representation of the biometric, and dedupe the first and second records. In some examples, an identification system may be used to determine an identity, locate a first record in the first system associated with the identity, locate a second record in a second system associated with the identity, and dedupe the first and second records. In a number of examples, an identification system may be used to locate a first record in a first system using identity information, locate a second record in a second system using the identity information, determine that the first record and the second record cannot be verified as associated with a same person within a threshold certainty, and determine whether a person associated with the first record is associated with the second record using a digital representation of a biometric for the person.

In various embodiments, a system for integrating distributed systems includes at least one non-transitory storage medium that stores instructions and at least one processing unit. The at least one processing unit executes the instructions to obtain a digital representation of a biometric, determine an identity for a first system using the digital representation of the biometric, locate a first record in the first system associated with the identity using the digital representation of the biometric, locate a second record in a second system using the digital representation of the biometric, and dedupe the first record and the second record.

In some examples, an entity controls the first system and the second system. In other examples, a first entity controls the first system and a second entity controls the second system.

In a number of examples, the first record is associated with a first biometric template of a first type, the second record is associated with a second biometric template of a second type, and the at least one processing unit performs biometric template translation in order to compare the first biometric template to the second biometric template. In various examples, the at least one processing unit provides identity information obtained using the identity to the first system. In some examples, the at least one processing unit copies information from the second record to the first record as part of deduping the first record and the second record. In a number of examples, the at least one processing unit removes information from the first record as part of deduping the first record and the second record.

In some embodiments, a system for integrating distributed systems includes at least one non-transitory storage medium that stores instructions and at least one processing unit. The at least one processing unit executes the instructions to obtain a digital representation of a biometric, determine an identity using the digital representation of the biometric, locate a first record in a first system associated with the identity, locate a second record in a second system associated with the identity, and dedupe the first record and the second record.

In various examples, the at least one processing unit obtains identity information using the identity and locates the first record using the identity information. In some implementations of such examples, the at least one processing unit locates the second record using the identity information.

In a number of examples, the at least one processing unit locates the first record by creating a new record and dedupes the first record and the second record by copying information from the second record to the new record. In various examples, the first record and the second record are medical records. In some examples, the at least one processing unit obtains permission of a person associated with the identity before exchanging information between the first record and the second record.

In a number of embodiments, a system for integrating distributed systems includes at least one non-transitory storage medium that stores instructions and at least one processing unit. The at least one processing unit executes the instructions to use identity information to locate a first record in a first system, use identity information to locate a second record in a second system, determine that the first record and the second record cannot be verified as associated with a same person within a threshold certainty, obtain a digital representation of a biometric for a person associated with the first record, and use the digital representation of the biometric to determine whether the person is associated with the second record.

In some examples, the at least one processing unit dedupes the first record and the second record upon determining that the person is associated with the second record. In various examples, the at least one processing unit links the first record and the second record upon determining that the person is associated with the second record. In a number of examples, the at least one processing unit requests the person for the digital representation of the biometric. In various examples, the identity information is at least one of a name, an address, a birth date, or an identification number.

In a number of examples, the digital representation of the biometric is a first biometric template of a first type and the second record is associated with a second biometric template of a second type. In some implementations of such examples, the at least one processing unit translates the first biometric template in order to compare the first biometric template to the second biometric template.

### BRIEF DESCRIPTION OF THE DRAWINGS

The disclosure will be readily understood by the following detailed description in conjunction with the accompanying drawings, wherein like reference numerals designate like structural elements.

FIG. 1 depicts a first example system for integrating distributed systems.

FIG. 2 depicts a first example method for integrating distributed systems. This method may be performed by the system of FIG. 1.

FIG. 3 depicts a second example method for integrating distributed systems. This method may be performed by the system of FIG. 1.

FIG. 4 depicts a third example method for integrating distributed systems. This method may be performed by the system of FIG. 1.

FIG. 5 depicts a fourth example method for integrating distributed systems. This method may be performed by the system of FIG. 1.

FIG. 6 depicts a fifth example method for integrating distributed systems. This method may be performed by the system of FIG. 1.

FIG. 7 depicts a sixth example method for integrating distributed systems. This method may be performed by the system of FIG. 1.

FIG. 8A depicts a second example system for integrating distributed systems.

FIG. 8B depicts the system of FIG. 8A upon biometric identification of the person.

FIG. 9 depicts a first example system for biometric template translation.

FIG. 10 depicts an example of generating a biometric template from a biometric.

FIG. 11 is a flow chart illustrating a first example method for biometric template translation. This method may be performed by the systems of FIG. 1, 9, or 13.

FIG. 12 is a flow chart illustrating a second example method for biometric template translation. This method may be performed by the systems of FIG. 1, 9, or 13.

FIG. 13 depicts a second example system for biometric template translation.

FIG. 14 is a flow chart illustrating a third example method for biometric template translation. This method may be performed by the systems of FIG. 1, 9, or 13.

FIG. 15 is a flow chart illustrating a fourth example method for biometric template translation. This method may be performed by the systems of FIG. 1, 9, or 13.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

The description that follows includes sample systems, methods, apparatuses, and computer program products that embody various elements of the present disclosure. However, it should be understood that the described disclosure may be practiced in a variety of forms in addition to those described herein.

Many different systems and/or devices in such systems may store records about people. Such records may be medical, financial, informational, and so on. Multiple different records may exist in separate systems and/or devices in such systems that relate to the same person. In some situations, the records may be unrelated and/or not duplicative of each other. However, in other situations, the records may be related. In such situations, the records may be duplicative of each other, inconsistent, and so on.

In some scenarios, the same entity may control the different individual systems. For example, a medical network may include a number of different medical systems associated with a number of different medical providers, including a general practitioner doctor and a podiatrist. Thus, the medical provider network may include a first record about a person in the general practitioner's system created when the person first visited the general practitioner and a second record about the person in the podiatrist's system created when the person first visited the podiatrist. The first and second records may be duplicative in that they may contain repeated recordings of some of the same information about the person. The first and second records may also be inconsistent, such as where the first record has a phone number for the person and the second record does not, the first record includes an updated mailing address for the person and the second record includes an out of date mailing address for the person, the first record and the second record include information related to services respectively provided by the general practitioner or podiatrist and entered into the respective record in their own system but not provided to the other despite possibly being relevant to medical services provided to the person by the other, and so on.

As a result, the distributed systems and/or components thereof may not be capable of performing functions related to accessing all of the information that is disparately, possibly inaccurately in some situations, stored. Further, the distributed systems and/or components thereof may consume excess resources and/or otherwise operate inefficiently for storing duplicative information, obtaining and storing information that has already been obtained, and so on.

The situation may be similar for distributed systems involving individual systems that are controlled by different entities. In such a scenario, the above issues may be exacerbated by the different procedures and/or mechanisms by which the individual systems obtain information, store information, use information, and so on.

The present disclosure relates to integrating distributed systems. One or more digital representations of biometrics may be used to match and/or dedupe first and second records respectively stored in first and second systems. Deduping may involve removing duplicate information from one or more of the records and/or the records themselves, linking the records, copying information from one record to the other, and so on. In various examples, an identification system may be used to determine an identity for a first system using a digital representation of a biometric, locate a first record in the first system associated with the identity using the digital representation of the biometric, locate a second record in a second system associated with the identity using the digital representation of the biometric, and dedupe the first and second records. In some examples, an identification system may be used to determine an identity, locate a first record in the first system associated with the identity, locate a second record in a second system associated with the identity, and dedupe the first and second records. In a number of examples, an identification system may be used to locate a first record in a first system using identity information, locate a second record in a second system using the identity information, determine that the first record and the second record cannot be verified as associated with a same person within a threshold certainty, and determine whether a person associated with the first record is associated with the second record using a digital representation of a biometric for the person.

In this way, the first and second systems and/or another system that communicates with and/or includes the first and second systems may be capable of performing functions related to accessing all of the information that is disparately, possibly inaccurately in some situations, stored. Further, the first and second systems and/or another system that communicates with and/or includes the first and second systems may consume excess resources and/or otherwise operate inefficiently for storing duplicative information, obtaining and storing information that has already been obtained, and so on.

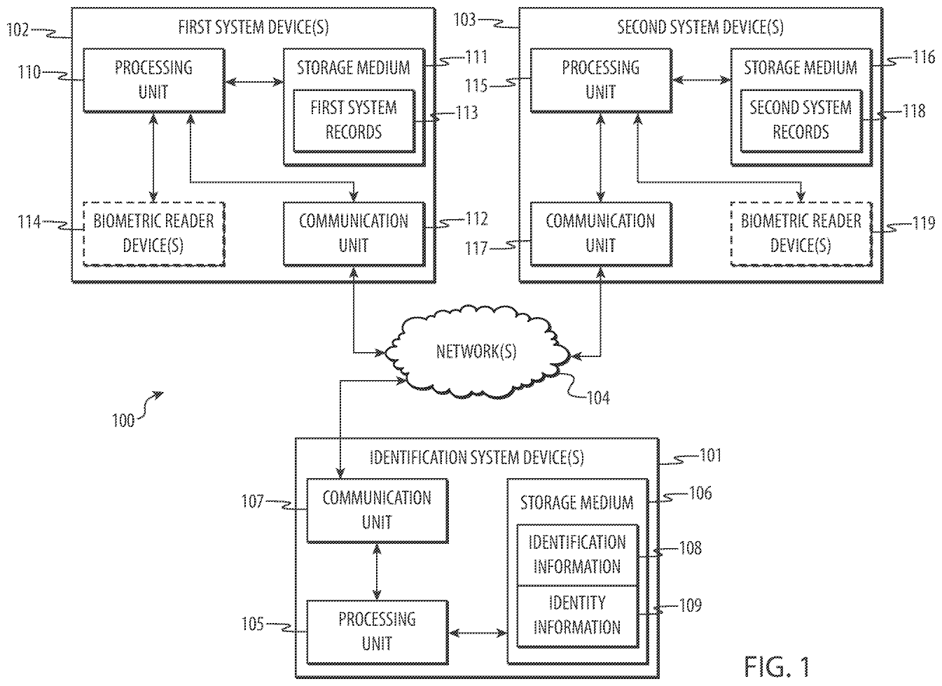

These and other embodiments are discussed below with reference to FIGS. 1-15. However, those skilled in the art will readily appreciate that the detailed description given herein with respect to these Figures is for explanatory purposes only and should not be construed as limiting.

FIG. 1 depicts a first example system 100 for integrating distributed systems. The system 100 may include one or more identification system devices 101 that are operable to communicate with one or more first system devices 102 and/or one or more second system devices 103 via one or more networks 104.

The identification system device 101 may be operative to provide identification services and/or identification related services to the first system device 102 and/or the second system device 103. For example, the identification system device 101 may store identity information 109 associated with identification information 108 (such as biometric data, logins, passwords, account identifiers, and so on) and may use the identification information 108 to control access to the identity information 109. For example, the identification system device 101 may be operative to receive one or more digital representations of biometrics, determine one or more identities by comparing the digital representations of biometrics to stored biometric data associated with the identity information 109, and provide information about the determined identities (such as identifying the identity, providing information stored in the identity information 109, providing attestations about the identity, and so on).

For example, the first system device 102 and/or the second system device 103 may be medical provider systems. The respective medical providers associated with the first system device 102 and/or the second system device 103 may use the identification system device 101 to identify patients upon check in for medical services, to track patients during various stages of medical procedures, identify medical provider personnel, track and/or control access of patients and/or medical provider personnel to secured areas (such as procedure rooms, equipment, supplies, and so on), and so on.

Additionally, the identification system device 101 may be operative to perform one or more methods to integrate distributed systems, such as one or more systems including the first system device 102 and/or the second system device 103. For example, first system records 113 may be included in the first system device 102 and second system records 118 may be included in the second system device 103. In some situations, the first system records 113 may include a first record for a person and the second system records 118 may include a second record for the person. The identification system device 101 may be operative to integrate distributed systems by deduping one or more of the records. Deduping may involve removing duplicate information from one or more of the records and/or the records themselves, linking the records, copying information from one record to the other, and so on. In various implementations, the identification system device 101 may use one or more digital representations of biometrics to match and/or dedupe the first and second records respectively stored by the first system device 102 and the second system device 103.

For example, the identification system device 101 may receive a digital representation of a biometric for a person to determine an identity of the person for the first system device 102. The identification system device 101 may locate a first record in the first system device 102 associated with the identity using the digital representation of the biometric. The identification system device 101 may also locate a second record in the second system device 103 associated with the identity using the digital representation of the biometric. For example, the first and second records may include and/or otherwise be associated with biometric data that matches and/or substantially matches the digital representation of the biometric. As such, the identification system device 101 may locate the first and second records by searching and/or otherwise querying the first system device 102 and/or the second system device 103 using the digital representation of the biometric and/or other hash or derivation thereof. The identification system device 101 may then dedupe the first and second records.

The above is described in the context of the identification system device 101 using the digital representation of the biometric to locate the first and/or second records. However, it is understood that this is an example. In various implementations, identity information 109 (such as one or more names, addresses, email addresses, phone numbers, patient identifiers, social security numbers, and so on) associated with the identity may be used as a search key into records stored by the first system device 102 and/or the second system device 103 instead of and/or in addition to the digital representation of the biometric, and/or hashes and/or other derivations of combinations of the identity information 109 and/or the digital representation of the biometric. Various configurations are possible and contemplated without departing from the scope of the present disclosure.

For example, the identification system device 101 may obtain a digital representation of a biometric of a person and use the digital representation of the biometric to determine an identity of the person. The identification system device 101 may locate a first record in the first system device 102 associated with the identity. The identification system device 101 may also locate a second record in the second system device 103 associated with the identity. The identification system device 101 may then dedupe the first and second records.

In some implementations, the identification system device 101 may perform such processes in order to dedupe records stored by the first system device 102 and/or the second system device 103 upon receiving a request to determine an identity for the first system device 102 and/or the second system device 103, upon receiving a digital representation of a biometric, and so on. However, it is understood that these are examples.

In other implementations, the identification system device 101 may use artificial intelligence to analyze records stored by the first system device 102 and/or the second system device 103 in order to attempt to dedupe one or more records. By way of illustration, the identification system device 101 may compare information contained in such records to attempt to determine whether or not the records correspond to the same person (such as by comparing one or more such names, addresses, email addresses, phone numbers, patient identifiers, social security numbers, combinations thereof, hashes and/or other derivations thereof, and so on).

In some situations, the artificial intelligence used by the identification system device 101 may determine that there is a possibility that a first record and a second record may correspond to the same person. The identification system device 101 may determine a threshold certainty (such as a 51% threshold certainty, a 95% threshold certainty, a 99% threshold certainty, and so on) of whether or not the first and second records do, in fact, correspond to the same person. If the threshold certainty is not met, the identification system device 101 may wait to dedupe the first and second records until a digital representation of a biometric for the person related to one or more of the records can be obtained and used to verify whether or not the first and second records are actually for the same person. In some implementations, the identification system device 101 may take no further action on the first and the second records until the digital representation of the biometric is received for purposes of an identity determination. In other examples, the identification system device 101 may request the digital representation of the biometric (such as by transmitting a request to an electronic device, telephone number, email address, and/or other contact information associated with the person in the identity information 109). Various configurations are possible and contemplated without departing from the scope of the present disclosure.

The identification system device 101 may be set for a particular threshold certainty for different uses. For example, an advertising targeting use case may be allowed to accept matches having as low as a 51% threshold certainty. However, a medical records matching use case may necessitate a much higher threshold certainty, such as 99%, to avoid harm due to incorrect matching. Various configurations are possible and contemplated without departing from the scope of the present disclosure.

By way of illustration, the identification system device 101 may determine that there may be a 99% certainty that two records listing a name of “Zeblum Xanadex,” with a city of “Yuma, Nebr.,” and a birthdate of “Feb. 29, 2016” are both about the same person due to the relatively uncommon name and birth date of people in that town whereas there may be only a 40% certainty that two records listing a name of “Mary O'Reilly,” with a city of “Boston, Mass.,” and a birthdate of “Nov. 1, 1978” are both about the same person due to the relatively common name and birth date of people in that city. As such, the identification system device 101 may dedupe the two records listing the name of “Zeblum Xanadex” but wait to dedupe the two records listing the name of “Mary O'Reilly” until a digital representation of a biometric for a person associated with one of the two records can be obtained and used to determine whether or not the two records relate to the “Mary O'Reilly.” Various configurations are possible and contemplated without departing from the scope of the present disclosure.

For example, the identification system device 101 may locate a first record in the first system device 102 using the identity information 109. The identification system device 101 may locate a second record in a second system using the identity information 109. The identification system device 101 may determine that the first record and second record cannot be verified as associated with a same person within a threshold certainty. As such, the identification system device 101 may determine whether a person associated with the first record is associated with the second record using a digital representation of a biometric for the person.

In these ways, the system 100 may be capable of performing functions related to accessing all of the information that may be disparately, possibly inaccurately in some situations, stored. Further, the system 100 may consume excess resources and/or otherwise operate inefficiently for storing duplicative information, obtaining and storing information that has already been obtained, and so on.

In some implementations, the digital representation of the biometric and/or biometric data stored by the identification system device 101, the first system device 102, and/or the second system device 103 may be biometric templates that are of different types and/or formatted differently. In such implementations, comparison of the digital representation of the biometric and/or biometric data stored by the identification system device 101, the first system device 102, and/or the second system device 103 may not be possible in some situations without performing biometric template translation. In such situations, biometric template translation may be performed by the identification system device 101 and/or another device in order to compare the digital representation of the biometric and/or biometric data stored by the identification system device 101, the first system device 102, and/or the second system device 103. This and techniques of biometric template translation will be discussed and elaborated further below.

The identification system device 101 may be any kind of electronic device and/or cloud and/or other computing arrangement and may include one more processing units 105 and/or other processors or controllers, non-transitory storage media 106 (which may take the form of, but is not limited to, a magnetic storage medium; optical storage medium; magneto-optical storage medium; read only memory; random access memory; erasable programmable memory; flash memory; and so on), communication units 107, and/or other components. The processing unit 105 may execute one or more sets of instructions stored in the non-transitory storage medium 106 to perform various functions, such as storing biometric data and/or other identification information 108 for people and associated identity information 109 (such as one or more names, addresses, telephone numbers, financial data, financial account numbers, verified ages, insurance identifiers, payment account identifiers, and so on), receiving one or more digital representations of biometrics, matching one or more received digital representations of biometrics to stored biometric data, retrieving identity information associated with stored biometric data matching one or more received digital representations of biometrics, providing retrieved identity information, communicating with the first system device 102 and/or the second system device 103 via the network 104 using the communication unit 107, matching records stored by the first system device and/or the second system device 103, deduping records stored by the first system device 102 and/or the second system device 103, translating between one or more types of biometric templates, and so on.

Likewise, the first system device 102 and/or the second system device 103 may be any kind of electronic devices. Examples of such devices include, but are not limited to, one or more desktop computing devices, laptop computing devices, mobile computing devices, wearable devices, tablet computing devices, mobile telephones, smart phones, printers, displays, kiosks, vehicles, kitchen appliances, entertainment system devices, digital media players, and so on. The devices may include one or more processing units 110, 115 and/or other processors and/or controllers; one or more non-transitory storage media 111, 116; one or more communication units 112, 117; and so on. In some implementations, the first system device 102 and/or the second system device 103 may include one or more biometric reader devices 114, 119 (such as a fingerprint scanner, a blood vessel scanner, a palm-vein scanner, an optical fingerprint scanner, a phosphorescent fingerprint scanner, a still image and/or video camera, a 2D and/or 3D image sensor, a capacitive sensor, a saliva sensor, a deoxyribonucleic acid sensor, a heart rhythm monitor, a microphone, and so on). In other implementations, the first system device 102 and/or the second system device 103 may instead communicate with one or more other devices that include one or more biometric reader devices 114, 119. The processing units 110, 115 may respectively execute one or more sets of instructions stored in the non-transitory storage media 111, 116 to perform various functions, such as using the biometric reader devices 114, 119 to obtain one or more digital representations of one or more biometrics (such as one or more hashes and/or other digital representations of one or more fingerprints, blood vessel scans, palm-vein scans, voiceprints, facial images, retina images, iris images, deoxyribonucleic acid sequences, heart rhythms, gaits, and so on) for a person, storing (and/or maintaining, updating, analyzing, deleting, and so on) one or more records (such as the first system records 113 stored in the storage medium 111, the second system records 118 stored in the storage medium 116, and so on), communicating with the identification system device 101 and/or each other via the network 104 using the communication units 112, 117, providing one or more obtained digital representations of biometrics, and so on.

Although the system 100 is illustrated and described as including particular components configured in a particular arrangement, it is understood that this is an example. By way of illustration, the system 100 is illustrated and described as the first system device 102 including the biometric reader device 114 and the second system device 103 including the biometric reader device 119. However, in other implementations, the biometric reader device 114 and/or the biometric reader device 119 may be included in another electronic device that is operative to communicate with the identification system device 101, the first system device 102, and/or the second system device 103. Various configurations are possible and contemplated without departing from the scope of the present disclosure.

By way of another example, in some implementations, the first system device 102 may be configured to communicate with the identification system device 101 and the second system device 103 may not be configured to communicate with the identification system device 101. In such an implementation, the first system device 102 may be configured to communicate with the identification system device 101 to obtain identification information that may be used to dedupe records stored in association with the second system device 103 and the first system device 102 may then use that identification information to communicate with the second system device 103 directly. For example, the first system device 102 may be configured to communicate with the identification system device 101 to identify a person using a biometric template of a first type, retrieve a biometric template of a second type that is associated with the identity and used by the second system device 103 from the identification system device 101, and communicate the biometric template of the second type to the second system device 103 as part of a records request. Various implementations are possible and contemplated without departing from the scope of the present disclosure.

By way of yet another example, the above illustrates and describes information regarding records passing through the identification system device 101. However, it is understood that this is an example. In various implementations, the identification system device 101 may be used solely for identification to the first system device 102 and/or the second system device 103 and these devices (and/or one or more centralized records repositories) may then communication various information from one or more records directly (which may be in response to authorizations received from the identification system device 101) rather than sending such information through the identification system device. Various implementations are possible and contemplated without departing from the scope of the present disclosure.

In yet another example, the above illustrates and describes association of records with a single identity. However, it is understood that this is an example. In some implementations, a number of records may be associated with a group of people. For example, family medical histories may be linked to the identities and/or biometric data associated with multiple members of a family. In such an example, deduping may involve following these associated links and/or using biometric data to identify and/or authorize access to information associated therewith (such as obtaining access to a family medical history using linked biometric data of the various family members). Various implementations are possible and contemplated without departing from the scope of the present disclosure.

By way of still another example, the above is illustrated and described as deduping and/or authorizing access using biometric data of the person associated with the record. However, in various implementations, biometric data of a person may be evaluated for access and/or linking to information in records to which that person has access. For example, a doctor may provide biometric data to request patient records, verify to patients that it is their doctor requesting their records, identify records of patients associated with the doctor, and so on. Various implementations are possible and contemplated without departing from the scope of the present disclosure.

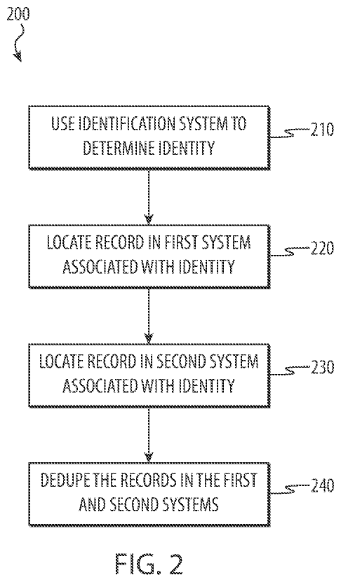

FIG. 2 depicts a first example method 200 for integrating distributed systems. This method 200 may be performed by the system 100 of FIG. 1.

At operation 210, an electronic device (such as the identification system device 101 of FIG. 1, the first system device 102 of FIG. 1, and so on) may use an identification system to determine an identity for a person. For example, a digital representation of a biometric may be obtained from the person. The digital representation of the biometric may be compared to stored biometric data and/or other identification information that is associated with identity information. Upon determining the identity (e.g., the digital representation of the biometric matches stored biometric data), information from the identity information may be returned.

At operation 220, the electronic device may locate a first record in a first system that is associated with the identity. At operation 230, the electronic device may locate a second record in a second system that is associated with the identity.

The first record may be located and/or otherwise accessed in the first system by comparing the digital representation of the biometric and/or the stored biometric data to biometric data associated with the first record. Alternatively, information from the identity information (and/or combinations and/or hashes or other derivations thereof, combinations of the information and the digital representation of the biometric and/or the stored biometric data, and so on) may be used as a key to search for records with matching and/or substantially matching information in the first system. The second record in the second system may be similarly located and/or otherwise accessed.

At operation 240, the electronic device may dedupe the first and second records. Deduping may involve removing duplicate information from one or more of the first and second records and/or the first and second records themselves, linking the first and second records, copying information from one of the first and second records to the other, and so on.

For example, the first and second records may be different medical records in different medical provider systems for the same person. A person may have a medical record at an internal medicine provider's office, but may not have previously used an identification system to check in for an appointment there. The person may then provide a digital representation of a biometric to check in for an appointment. The digital representation of the biometric may be used to determine an identity for the person and identity information for that person (such as the person's name, birth date, address, phone number, a hash or other derivation of a combination of these, and so on) may be used to locate the medical record for the person in an internal medicine provider system. The digital representation of the biometric may also be used to locate other systems accessible to the identification system that have records associated with matching biometric data, such as a medical record in a chiropractic medicine provider system. The medical record in the internal medicine provider system and the medical record in the chiropractic medicine provider system may be deduped.

By way of illustration, the records may be deduped by copying information from one record to another when the records include different information, such as where one record has a phone number for the person but the other record does not. Alternatively and/or additionally, the two records may be linked so that future updates can be applied to only one of the records to avoid storing duplicate information while still being accessible via both, such as where the internal medicine provider medical record will get updates for newly performed medical procedures listed in the chiropractic medicine provider record. Alternatively and/or additionally, information may be removed from one of the two records (and/or one of the records may be removed) and replaced with a link to the other to avoid storing duplicate information while allowing the information to remain accessible via the two systems. Various configurations are possible and contemplated without departing from the scope of the present disclosure.

In some situations, deduping the first and second records may involve exchange of and/or access to restricted information that may require patient approval (such as information regulated by the Health Insurance Portability and Accountability Act). In such a situation, information exchange and/or access may be performed according to patient instructions and/or permissions specified in the identity information and/or the records. Alternatively, the person may be contacted for permission before exchange and/or access (such as by transmitting a message to the person via contact information specified in the identity information, like a text message transmitted to a mobile telephone associated with the person where the person may reply to the text message to approve).

In various examples, this example method 200 may be implemented as a group of interrelated software modules or components that perform various functions discussed herein. These software modules or components may be executed within a cloud network and/or by one or more computing devices, such as the identification system device 101 of FIG. 1.

Although the example method 200 is illustrated and described as including particular operations performed in a particular order, it is understood that this is an example. In various implementations, various orders of the same, similar, and/or different operations may be performed without departing from the scope of the present disclosure.

For example, the method 200 is illustrated and described as locating a record in the first system that is associated with the identity. However, in some examples, the first system may not include a record that is associated with the identity. In such an example, locating the record in the first system may include creating a new, blank record. The new record may then be associated with the identity and deduping may involve populating the new record with information from a record in the second system. Various configurations are possible and contemplated without departing from the scope of the present disclosure.

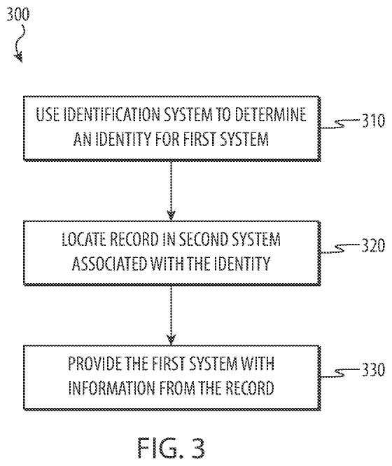

FIG. 3 depicts a second example method 300 for integrating distributed systems. This method 300 may be performed by the system 100 of FIG. 1.

At operation 310, an electronic device (such as the identification system device 101 of FIG. 1) may use an identification system to determine an identity of a person for a first system. At operation 320, the electronic device may locate a record in a second system associated with the identity.

For example, the electronic device may use the identification system to determine the identity by providing a digital representation of a biometric for the person to the identification system. The electronic device, and/or the identity system, may also use the digital representation of the biometric to search other accessible systems for records that are associated with biometric data matching and/or substantially matching the digital representation of the biometric.

At operation 330, the electronic device may provide the first system with information from the record. The information may be used to dedupe the record and a record stored by the first system.

For example, the electronic device may be a check in station in a doctor's office. The check in station may obtain a digital representation of a biometric for a person, use the identification system to determine an identity for the person using the digital representation of the biometric, and provide information about that identity to a doctor's office system to inform the doctor's office of the person's arrival for an appointment. The check in station may also use the digital representation of the biometric to locate medical records in other systems for the person that are associated with the identity and communicate information about those medical records to the doctor's office system. Various configurations are possible and contemplated without departing from the scope of the present disclosure.

In various examples, this example method 300 may be implemented as a group of interrelated software modules or components that perform various functions discussed herein. These software modules or components may be executed within a cloud network and/or by one or more computing devices, such as the identification system device 101 of FIG. 1.

Although the example method 300 is illustrated and described as including particular operations performed in a particular order, it is understood that this is an example. In various implementations, various orders of the same, similar, and/or different operations may be performed without departing from the scope of the present disclosure.

For example, the method 300 is illustrated and described as providing the first system with information from the record in the second system. However, it is understood that this is an example. In various implementations, the electronic device may provide the second system with information from a record in the first system associated with the identity (such as for the purpose of updating the record in the second system) instead of and/or in addition to providing the first system with information from the record in the second system. Various configurations are possible and contemplated without departing from the scope of the present disclosure.

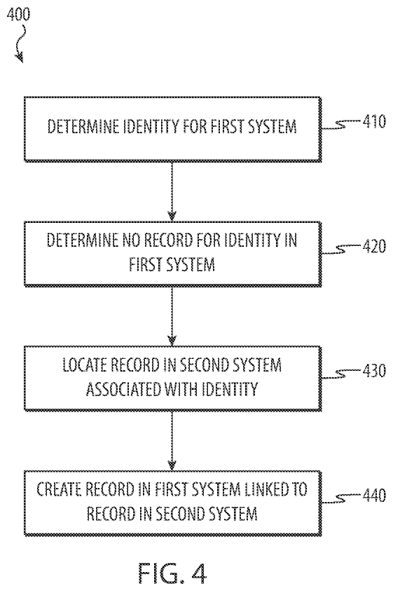

FIG. 4 depicts a third example method 400 for integrating distributed systems. This method 400 may be performed by the system 100 of FIG. 1.

At operation 410, an electronic device (such as the identification system device 101 of FIG. 1) may determine an identity of a person for a first system. At operation 420, the electronic device may determine that there is no record associated with the identity in the first system. At operation 430, the electronic device may locate a record in a second system associated with the identity. At operation 440, the electronic device may create a record in the first system that is linked to the record in the second system.

For example, the electronic device may be a check in station in a doctor's office. A person may use the check in station, asserting that the person has an appointment at the doctor's office and has registered with an identification system. The check in station may obtain a digital representation of a biometric from the person and use such with the identification system to determine the person's identity. Thereupon, the electronic device may determine that the identity is associated with an appointment in the doctor's office system, but the doctor's office system does not yet have a medical record for the person. The electronic device may use the digital representation of the biometric and/or information associated with the identity to look up other systems where medical records associated with the person are stored. The electronic device may then create a medical record for the person in the doctor's office system that is linked to the located medical records in the other systems, functionally forming a unified medical record for the person including all of the located records. As such, the doctor's office may be able to access the unified medical record for the person without requiring the person to provide all of that information directly to the doctor's office.

In various examples, this example method 400 may be implemented as a group of interrelated software modules or components that perform various functions discussed herein. These software modules or components may be executed within a cloud network and/or by one or more computing devices, such as the identification system device 101 of FIG. 1.

Although the example method 400 is illustrated and described as including particular operations performed in a particular order, it is understood that this is an example. In various implementations, various orders of the same, similar, and/or different operations may be performed without departing from the scope of the present disclosure.

For example, the method 400 is illustrated and described as linking the record in the first system to the record in the second system. However, it is understood that this is an example. In some implementations, information from the record in the second system may instead be copied to the record in the first system. Various configurations are possible and contemplated without departing from the scope of the present disclosure.

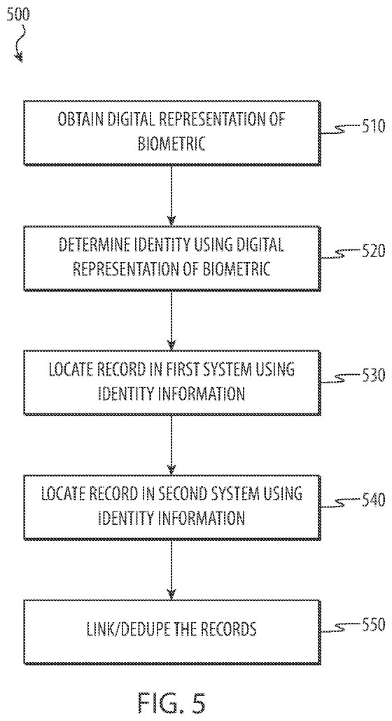

FIG. 5 depicts a fourth example method 500 for integrating distributed systems. This method 500 may be performed by the system 100 of FIG. 1.

At operation 510, an electronic device (such as the identification system device 101 of FIG. 1) may obtain a digital representation of a biometric for a person. At operation 520, the electronic device may determine an identity of the person using the digital representation of the biometric.

At operation 530, the electronic device may locate a record in a first system using the identity information. At operation 540, the electronic device may locate a record in a second system using the identity information.

For example, the electronic device may use information from the identity information as a key to search for records including that information in the first and second systems. Such information that may be used as a search key includes one or more names, telephone numbers, addresses, email addresses, social security numbers, patient identifiers, driver's license and/or other identification numbers, license plate numbers, biometric data, and so on. In some implementations, combinations of this information and/or the digital representation of the biometric may be used. In various implementations, the information and/or combinations of the information may be hashed, encrypted, and/or otherwise processed to reduce search time, control access to the information, and so on. Various configurations are possible and contemplated without departing from the scope of the present disclosure.

At operation 550, the electronic device may link and/or otherwise dedupe the records in the first and second systems. For example, by locating records in the first and second systems using the identity information, the electronic device may establish that the records are for the same person. As the electronic device determines that the records in the first and second systems are for the same person, the electronic device may link and/or otherwise dedupe the records in the first and the second systems. In this way, the electronic device may turn the disparate records into a universal record and/or a more universal record for the person.

In various examples, this example method 500 may be implemented as a group of interrelated software modules or components that perform various functions discussed herein. These software modules or components may be executed within a cloud network and/or by one or more computing devices, such as the identification system device 101 of FIG. 1.

Although the example method 500 is illustrated and described as including particular operations performed in a particular order, it is understood that this is an example. In various implementations, various orders of the same, similar, and/or different operations may be performed without departing from the scope of the present disclosure.

For example, the method 500 is illustrated and described as using the identity information to locate the records in the first and the second systems. However, it is understood that this is an example. In various implementations, other information may be used to search the first and/or second systems and/or otherwise access records in the first and/or the second systems. By way of illustration, in some implementations, the electronic device may use the digital representation of the biometric to search and/or otherwise access records in the first and/or second systems. Various configurations are possible and contemplated without departing from the scope of the present disclosure.

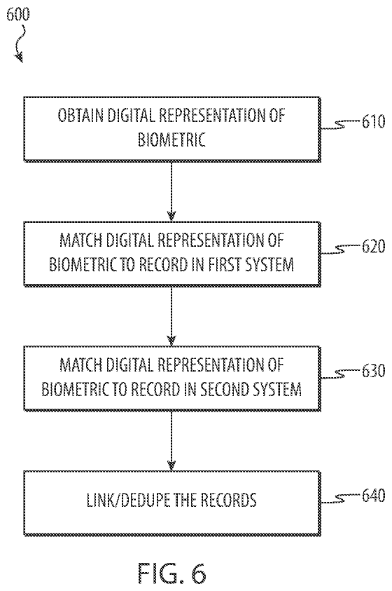

FIG. 6 depicts a fifth example method 600 for integrating distributed systems. This method 600 may be performed by the system 100 of FIG. 1.

At operation 610, an electronic device (such as the identification system device 101 of FIG. 1) may obtain a digital representation of a biometric for a person. At operation 620, the electronic device may match (and/or substantially match) the digital representation of the biometric to a record in a first system. For example, the electronic device may match the digital representation of the biometric to biometric data stored in and/or otherwise associated with the record in the first system. At operation 630, the electronic device may match (and/or substantially match) the digital representation of the biometric to a record in a second system. At operation 640, the electronic device may link and/or otherwise dedupe the records.

In various examples, this example method 600 may be implemented as a group of interrelated software modules or components that perform various functions discussed herein. These software modules or components may be executed within a cloud network and/or by one or more computing devices, such as the identification system device 101 of FIG. 1.

Although the example method 600 is illustrated and described as including particular operations performed in a particular order, it is understood that this is an example. In various implementations, various orders of the same, similar, and/or different operations may be performed without departing from the scope of the present disclosure.

For example, the method 600 is illustrated and described as comparing the digital representation of the biometric to biometric data stored in and/or otherwise associated with records in the first and/or second systems. However, in some implementations, the digital representation of the biometric and the biometric data may be biometric templates of different types (e.g., formatted differently). As such, biometric template translation may be performed to enable comparison between the digital representation of the biometric to biometric data stored in and/or otherwise associated with records in the first and/or second systems. Such biometric templates, types, formatting, and biometric template translation is discussed and elaborated in detail below.

Further, the above illustrates and describes obtaining a digital representation of a biometric and using such to determine that records in first and second systems correspond to the same person and should be linked and/or otherwise deduped. This is an example of a “just in time” approach where unique identifying information may be obtained and used to locate and evaluate records in different systems that may correspond to the same person. However, in other approaches, information in records in different systems may be evaluated and/or compared (such as using an artificial intelligence record and/or information matching software) to determine records that may correspond to the same person. Biometric identification may then be used to determine whether or not identified records correspond to the same person, and/or increase the accuracy of such a determination if a threshold certainty has not been achieved otherwise. Various configurations are possible and contemplated without departing from the scope of the present disclosure.

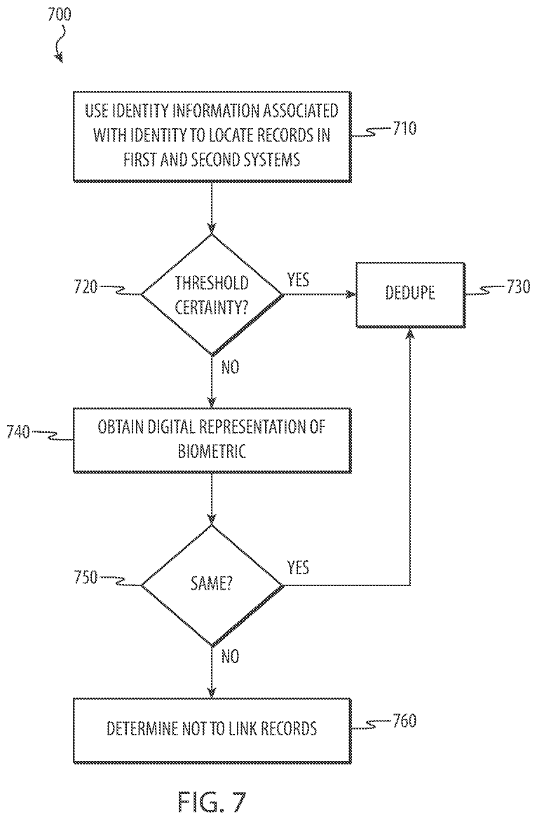

For example, FIG. 7 depicts a sixth example method 700 for integrating distributed systems. This method 700 may be performed by the system 100 of FIG. 1.

At operation 710, an electronic device (such as the identification system device 101 of FIG. 1) may use identity information associated with an identity to locate records in first and second systems that may correspond to a same person. The identity information may include one or more names, telephone numbers, addresses, birth dates, patient identifiers, driver's license and/or other identification numbers, social security numbers, combinations thereof, hashes or other derivations thereof and/or of combinations thereof, and so on.

At operation 720, the electronic device may determine whether or not the electronic device can determine within a threshold certainty (such as a 51% threshold certainty for low risk applications like advertising targeting, a 95% threshold certainty for higher risk applications like financial applications, a 99% threshold certainty for ultra-high risk applications such as medical applications, and so on) that the records correspond to the same person. If so, the flow may proceed to operation 730 where the electronic device may link and/or otherwise dedupe the records in the first and second systems. Otherwise, the flow may proceed to 740.

At operation 740, after the electronic device has determined that the electronic device could not verify that the records in the first and second systems corresponded to the same person within the threshold certainty, the electronic device may obtain a digital representation of a biometric for the person. At operation 750, the electronic device may use the digital representation of the biometric to determine whether or not the records in the first and second systems correspond to the same person (such as by comparing the digital representation of the biometric to biometric data stored in the records in the first and/or second systems, using the digital representation of the biometric to determine an identity and retrieve identity information that can be compared to the records in the first and/or second systems, and so on).

If so, the flow may proceed to operation 730 where the electronic device may link and/or otherwise dedupe the records in the first and second systems. Otherwise, the flow may proceed to 760 where the electronic device may determine not to link and/or otherwise dedupe the records in the first and second systems because the electronic device cannot sufficiently verify that the records in the first and second systems correspond to the same person.

For example, the electronic device may determine that there may be a 99% certainty that two records listing a name of “Zeblum Xanadex,” with a city of “Yuma, Nebr.,” and a birthdate of “Feb. 29, 2016” are both about the same person due to the relatively uncommon name and birth date of people in that town whereas there may be only a 40% certainty that two records listing a name of “Mary O'Reilly,” with a city of “Boston, Mass.,” and a birthdate of “Nov. 1, 1978” are both about the same person due to the relatively common name and birth date of people in that city. As such, the electronic device may dedupe the two records listing the name of “Zeblum Xanadex” but wait to dedupe the two records listing the name of “Mary O'Reilly” until a digital representation of a biometric for a person associated with one of the two records can be obtained and used to determine whether or not the two records relate to the “Mary O'Reilly.” Various configurations are possible and contemplated without departing from the scope of the present disclosure.

In various examples, this example method 700 may be implemented as a group of interrelated software modules or components that perform various functions discussed herein. These software modules or components may be executed within a cloud network and/or by one or more computing devices, such as the identification system device 101 of FIG. 1.

Although the example method 700 is illustrated and described as including particular operations performed in a particular order, it is understood that this is an example. In various implementations, various orders of the same, similar, and/or different operations may be performed without departing from the scope of the present disclosure.

For example, the method 700 is illustrated and described as obtaining the digital representation of the biometric when the electronic device cannot determine that the records in the first and second systems correspond to the same person within a threshold certainty. However, in some implementations, the electronic device may obtain the digital representation of the biometric to use in evaluating the records in the first and second systems regardless whether or not the electronic device makes any threshold certainty determination with respect to any non-biometric information in the records. In various examples, the electronic device may omit any threshold certainty determination. Various configurations are possible and contemplated without departing from the scope of the present disclosure.

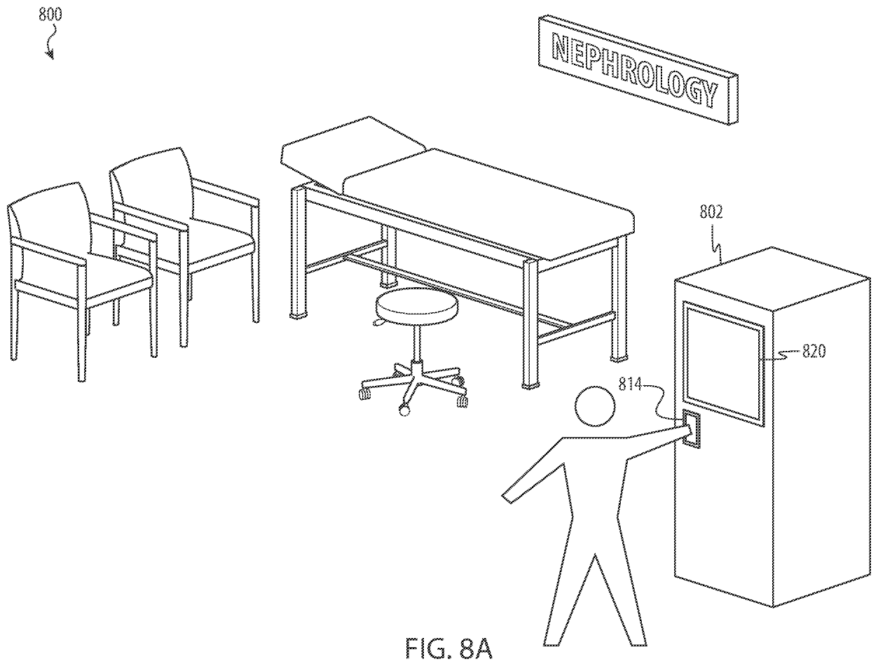

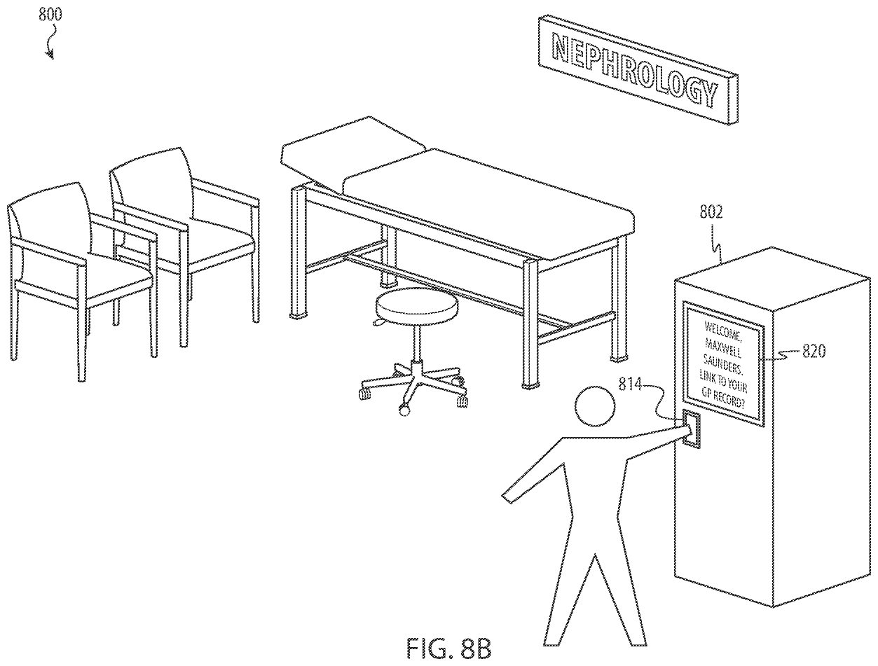

FIG. 8A depicts a second example system 800 for integrating distributed systems. In this example, a person may use a station 802 to check in for an appointment at a nephrologist's office by providing a fingerprint via a fingerprint scanner 814 of the station. The station 802 may determine an identity for the person using a digital representation of the fingerprint, locate a medical record for the person in the nephrologist's system, and determine whether or not any other medical records for the person can be located in any other systems accessible to the station 802 and/or another device with which the station 802 is operable to communicate. If so, the station 802 may prompt the person regarding whether or not to link such other records to the medical record for the person in the nephrologist's system.

For example, FIG. 8B depicts the system 800 of FIG. 8A upon biometric identification of the person. In this example, the station 802 has identified the person as “Maxwell Saunders” and presents a tailored welcome message to the person accordingly via an output device 820. Further in this example, the station 802 has located a medical record for the person in a general practitioner's system and presents a prompt to the person regarding whether or not to link the medical record for the person in the nephrologist's system with the medical record for the person in the general practitioner's system. Such linking may involve copying of information between the two records, removing information from one or more of the two records, linking the two records such that the information in one of the records is accessible via the other and/or changes to one of the records update and/or otherwise notify the other, and so on.

Although a particular configuration is illustrated and described, it is understood that this is an example. In other implementations, other configurations may be used. By way of illustration, in various implementations, the records in the nephrologist's system and/or the general practitioner's system may be analyzed to detect that they may both correspond to the person and the person may be requested to come in to provide the biometric in order to verify. Various configurations are possible and contemplated without departing from the scope of the present disclosure.

In various implementations, a system for integrating distributed systems may include at least one non-transitory storage medium that stores instructions and at least one processing unit. The at least one processing unit may execute the instructions to obtain a digital representation of a biometric, determine an identity for a first system using the digital representation of the biometric, locate a first record in the first system associated with the identity using the digital representation of the biometric, locate a second record in a second system using the digital representation of the biometric, and dedupe the first record and the second record.

In some examples, an entity may control the first system and the second system. In other examples, a first entity may control the first system and a second entity may control the second system.

In a number of examples, the first record may be associated with a first biometric template of a first type, the second record may be associated with a second biometric template of a second type, and the at least one processing unit may perform biometric template translation in order to compare the first biometric template to the second biometric template. In various examples, the at least one processing unit may provide identity information obtained using the identity to the first system. In some examples, the at least one processing unit may copy information from the second record to the first record as part of deduping the first record and the second record. In a number of examples, the at least one processing unit may remove information from the first record as part of deduping the first record and the second record.

In some implementations, a system for integrating distributed systems may include at least one non-transitory storage medium that stores instructions and at least one processing unit. The at least one processing unit may execute the instructions to obtain a digital representation of a biometric, determine an identity using the digital representation of the biometric, locate a first record in a first system associated with the identity, locate a second record in a second system associated with the identity, and dedupe the first record and the second record.

In various examples, the at least one processing unit may obtain identity information using the identity and locate the first record using the identity information. In some such examples, the at least one processing unit may locate the second record using the identity information.

In a number of examples, the at least one processing unit may locate the first record by creating a new record and dedupe the first record and the second record by copying information from the second record to the new record. In various examples, the first record and the second record may be medical records. In some examples, the at least one processing unit may obtain permission of a person associated with the identity before exchanging information between the first record and the second record.

In a number of implementations, a system for integrating distributed systems may include at least one non-transitory storage medium that stores instructions and at least one processing unit. The at least one processing unit may execute the instructions to use identity information to locate a first record in a first system, use identity information to locate a second record in a second system, determine that the first record and the second record cannot be verified as associated with a same person within a threshold certainty, obtain a digital representation of a biometric for a person associated with the first record, and use the digital representation of the biometric to determine whether the person is associated with the second record.

In some examples, the at least one processing unit may dedupe the first record and the second record upon determining that the person is associated with the second record. In various examples, the at least one processing unit may link the first record and the second record upon determining that the person is associated with the second record. In a number of examples, the at least one processing unit may request the person for the digital representation of the biometric. In various examples, the identity information may be at least one of a name, an address, a birth date, or an identification number.

In a number of examples, the digital representation of the biometric may be a first biometric template of a first type and the second record may be associated with a second biometric template of a second type. In some such examples, the at least one processing unit may translate the first biometric template in order to compare the first biometric template to the second biometric template.

As discussed above, various embodiments of the present disclosure may utilize biometric template translation. Techniques of biometric template translation will now be discussed and elaborated.

As part of biometric identification, biometric templates may be generated and provided to a matching engine that compares the biometric templates to stored biometric data. A biometric template may be generated by digitizing an obtained biometric (e.g., a biometric image or the like) and formatting the resulting digital data according to a particular format (e.g., a type of the biometric template) used by the matching engine. “Formatting,” as used herein, may mean cropping, skewing, hashing, compressing, mathematically expressing features and/or relationships among features, or otherwise manipulating part or all of a data set derived from the biometric, such as a biometric image. The stored biometric data may also be a biometric template formatted according to a particular format (e.g., type); the stored biometric data's type may or may not match that of the generated biometric template. This procedure may be performed for a variety of different kinds of biometrics. This may allow the matching engine to compare the biometric templates to the stored biometric data as long as both are configured according to a particular type (e.g., formatted in a particular way) expected by the matching engine.

Different matching engines (such as those provided by Innovatrics™, Chui™, Rank One Computing™, BioMoRF Systems Limited™, FotoNation™, and so on) may use different types of biometric templates (e.g., biometric templates that are formatted in different ways). Even for the same biometric (such as one or more fingerprints, blood vessel scans, palm-vein scans, voiceprints, facial images, retina images, iris images, deoxyribonucleic acid sequences, heart rhythms, gaits, and so on), images and/or other data may be cropped in different ways, features and/or relationships among features may be expressed differently mathematically, the same data may be represented in different portions, data may be hashed and/or compressed differently, and so on. As a result, biometric templates generated for one matching engine may not be capable of being compared to biometric data previously generated for another matching engine. This may cause different biometric systems to be incompatible, necessitating duplicate systems and/or components and/or excess processing and/or hardware and/or software resources.

In various implementations, a first biometric template of a first type is translated in order to enable the translated biometric template to be compared to a second biometric template of a second type. In one example, a canonical template structure to which the first and second biometric templates adhere may be identified and used to compare corresponding portions of the first and second biometric templates. In another example, types and associated formats of the first and second biometric templates may be identified to enable both to be translated to a general format that can then be compared. In still other examples, an identification system may use the first biometric template to determine an identity associated with first biometric data and retrieve second biometric data of a same type as the second biometric template. In still other examples, an identification system may use the first biometric template to determine an identity associated with first biometric data and verify that an account in an external system having a record storing the second biometric template is also associated with the identity, thus allowing translation between the first and second biometric templates through mutual association with the same identity. In this way, the biometric template translation may enable compatibility between different biometric systems. This may prevent duplication of systems and/or components, enable functions not previously performable, improve hardware and/or software performance of systems and/or components, and/or reduce consumption of hardware and/or software resources.

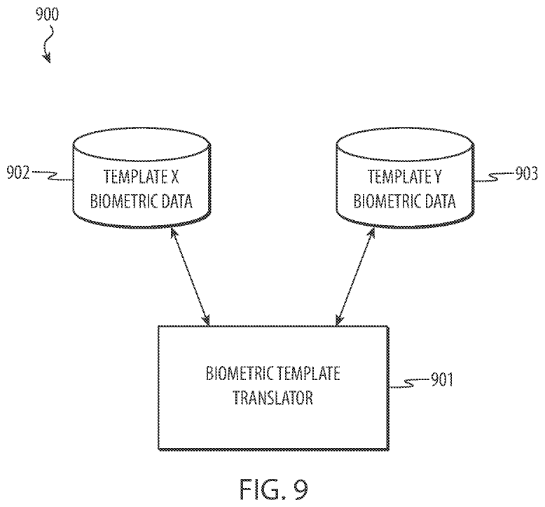

FIG. 9 depicts a first example system 900 for biometric template translation. The system includes a biometric template translator 901. The biometric template translator 901 may translate between biometric template of a first type and biometric template of a second type.

For example, the biometric template of the first type may be stored in a first data store 902 and the biometric template of the second type may be stored in a second data store 903. However, it is understood that this is an example. In various implementations, the biometric template of the first type and the biometric template of the second type may be stored in the same data store.

The biometric template of the first type and the biometric template of the second type may be generated for one or more different matching engines (such as Innovatrics™, Chui™ Rank One Computing™, BioMoRF Systems Limited™, FotoNation™, and so on) using one or more biometrics (such as one or more fingerprints, blood vessel scans, palm-vein scans, voiceprints, facial images, retina images, iris images, deoxyribonucleic acid sequences, heart rhythms, gaits, and so on). Both of the biometric template of the first type and the biometric template of the second type may include hashes and/or other digital representations of the biometrics. As the biometric template of the first type and the biometric template of the second type may be generated as different types, the biometric template of the first type and the biometric template of the second type may be generated and/or formatted differently. As the biometric template of the first type and the biometric template of the second type are of different types, they may not be comparable to each other until translated by the biometric template translator 901.

For example, the biometric template translator 901 may identify a canonical template structure to which both the first type and the second type adhere. For example, a canonical template structure may be a specification of how different types of templates all similarly format different portions of a biometric template. The canonical template structure may not specify how all portions of a biometric template are formatted, so different types of biometric templates may still be possible if the different types of biometric templates format the specified portions according to the canonical template structure but format other portions differently. Biometric templates may adhere to the canonical template structure if the biometric templates are formatted according to all of the specifications that are included in the canonical template structure. The biometric template translator 901 may use the canonical template structure to compare corresponding portions of the biometric template of the first type and the biometric template of the second type.

By way of another example, the biometric template translator 901 may identify the respective types of the biometric template of the first type and the biometric template of the second type. The biometric template translator 901 may determine a format that is associated with each of those types, use the determined formats to translate the biometric template of the first type and the biometric template of the second type to a general format version, and compare the general format versions. For example, each type of biometric template may be formatted in a particular way. By identifying the type of a biometric template, the biometric template translator 101 may able to determine how the data of that biometric template is formatted, convert that formatted data into a general format version, and then compare that general format version to other general format versions of biometric templates.

In yet another example, the biometric template translator 901 may use the biometric template of the first type to determine an identity associated with first biometric data in an identification system. The biometric template translator 901 may then translate between the biometric template of the first type and the biometric template of the second type by accessing stored biometric data of the second type associated with the identity and comparing that to the biometric template of the second type. Alternatively and/or additionally, the biometric template translator 901 may translate between the biometric template of the first type and the biometric template of the second type by verifying that an account in an external system having a record storing the biometric template of the second data store 903 is associated with the identity.

In yet another example, the biometric template translator 901 may use the biometric template of the first type to determine an identity associated with first biometric data in an identification system. For example, the identification system may identity people by matching received biometric templates to stored biometric data that is associated with identities. Further, the identification system may store biometric data of different types. Because the identification system stores the biometric data of the different types associated with the same identity, there is a certainty that the biometric data of the different types are all for the same person. When the identification system determines an identity by matching a biometric template to stored biometric data of one type, the identification system may be able to retrieve stored biometric data of other types that area also associated with the same identity. This may enable the identification system to provide access to stored biometric data of a particular desired type that is also associated with a identity after the identification system has determined the identity. As such, the biometric template translator 101 may translate between the biometric template of the first type and the biometric template of the second type by using the biometric template of the first type to determine an identity, accessing stored biometric data of the second type stored by the identification system associated with the identity, and comparing that to the biometric template of the second type. Alternatively and/or additionally, the biometric template translator 901 may verify that an account in an external system having a record storing the biometric template of the second type is associated with the determined identity, thus allowing translation between the first and second biometric templates through mutual association with the same identity.

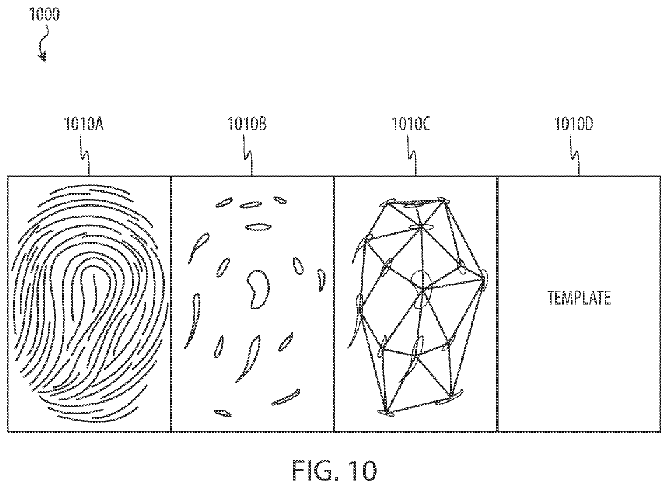

FIG. 10 depicts an example 1000 of generating a biometric template 1010D from a biometric data 1010A. In this example, a biometric data 1010A may be captured. A set of features 1010B may be identified from the biometric data 1010A. Relationships 1010C between the set of features 1010B may be identified, such as a map of relative differences and displacements between the set of features 1010B. A biometric template 1010D may then be generated by mathematically representing the relationships 1010C between the set of features 1010B.

Biometric templates 1010D of different types may be generated from biometrics 1010A. For example, different biometric templates 1010D may be generated by differently cropping an image of the biometric data 1010A and/or otherwise extracting data from different portions thereof, identifying different sets of features 1010B, differently mapping relationships 1010C between the set of features 1010B, differently mathematically representing the relationships 1010C between the set of features 1010B, differently formatting the mathematical representations of the relationships 1010C between the set of features 1010B, differently hashing and/or compressing data, and so on. Thus, different types of biometric templates 1010D may be generated from the same biometric data 1010A. The techniques of the present disclosure may be used to translate between these different types of biometric templates 1010D to enable comparison despite the different types (e.g., different formatting).

In FIG. 10, the biometric data 1010A is illustrated as a fingerprint and the relationships 1010C between the set of features 1010B are relative differences and displacements of data points of the fingerprint. However, it is understood that this is an example. Different biometrics, such as fingerprints, faces, irises, and so on may all have features (such as corners of a mouth or shapes of eyes) that may be identified from respective biometric data and mapped to compare relative differences and displacements. As such, relationships 1010C between sets of features 1010B for a variety of different biometric data 1010A may be mathematically represented in order to generate biometric templates 1010D of various different types or formats without departing from the scope of the present disclosure. The illustration of the biometric data 1010A in FIG. 2 as a fingerprint image is not intended to be limiting.

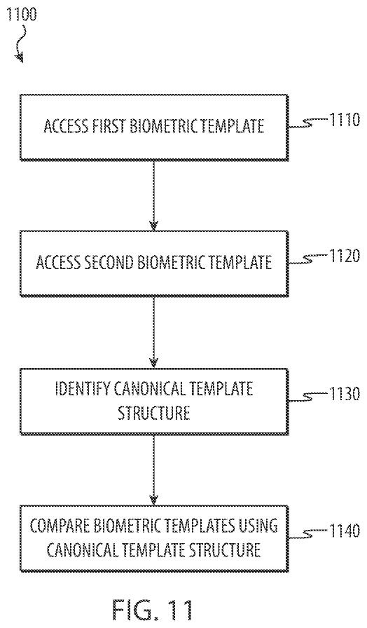

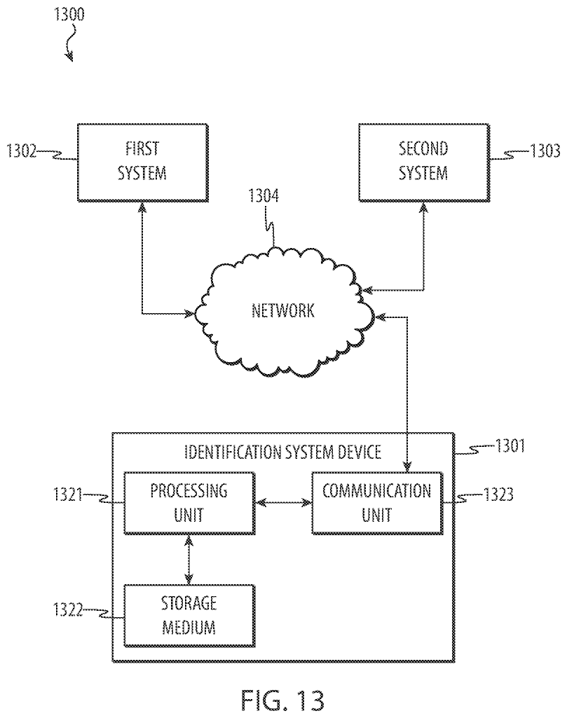

FIG. 11 is a flow chart illustrating a first example method 1100 for biometric template translation. This method 1100 may be performed by the systems 100, 900 of FIG. 1 or 9 (and/or the system 1300 of FIG. 13 discussed below).

At operation 1110, an electronic device (such as the identification system device 101 of FIG. 1, the biometric template translator 901 of FIG. 9, and/or the identification system device 1301 of FIG. 13 discussed below) may access a first biometric template. At operation 1120, the electronic device may access a second biometric template. The first and second biometric templates may be of different types. In other words, the first and second biometric templates may be formatted differently for different matching engines despite possibly being generated from the same biometric. For example, images and/or other data may be cropped in different ways, features and/or relationships among features may be expressed differently mathematically, the same data may be represented in different portions, data may be hashed and/or compressed differently, and so on.

At operation 1130, the electronic device may identify a canonical template structure. The canonical template structure may be one to which the first and second biometric templates both adhere. A canonical template structure may be a specification of how different types of templates all similarly format different portions of a biometric template. The canonical template structure may not specify how all portions of a biometric template are formatted, so different types of biometric templates may correspond to a single canonical template structure. For example, the different types of biometric templates may format specified portions according to the canonical template structure but format other portions differently. Put another way, biometric templates may adhere to the canonical template structure if the biometric templates are formatted according to all of the specifications that are included in the canonical template structure.

For example, the canonical template structure may be a standard according to which different biometric templates adhere. The standard may detail standard features of biometric templates that adhere to the canonical template structure. As such, all biometric templates that adhere to the canonical template structure may have particular corresponding portions. The standard may be a lightweight standard such that a great amount of variety is possible between different formats that all adhere to the standard.

Although the canonical template structure may be a standard according to which different templates adhere, that does not mean that all standards for biometric templates are canonical template structures. A standard for a biometric template may be a canonical template structure if identification of the fact that two biometric templates adhere to the standard allow the two biometrics to be compared using the standard. A standard for a biometric template may not be a canonical template structure if identification of the fact that two biometric templates adhere to the standard do not allow the two biometrics to be compared using the standard. Some standards may include more flexibility than allows for use of the standards for comparing biometric templates of different types. Such standards may not specify sufficient canonical structure to be a canonical template structures and be usable in this way.

For example, an American National Standards Institute (ANSI) standard for biometrics may require biometric templates to be formatted as packet data including a header packet indicating that the biometric templates are compliant with the ANSI standard and at least one biometric data packet that may optionally relate to a fingerprint, and iris, and so on. However, identification that a biometric template for a fingerprint and a biometric template for an iris are both compliant with the ANSI standard may not enable comparison of the biometric template for the fingerprint and the biometric template for an iris. The ANSI standard may also not require adherence to specifications for how details of biometrics are represented and formatted in the biometric data packet even when corresponding the same biometric.

At operation 1140, the electronic device may compare the first biometric template to the second biometric template using the canonical template structure. For example, the canonical template structure may be used to identify corresponding portions of the first and second biometric templates. The corresponding portions may then be compared.

If there is a match or substantial match, the electronic device may determine that the first and second biometric templates are from the same person. The electronic device may determine that the first and second biometric templates are from different people (and/or a determination that the first and second biometric templates are from the same person is not possible) when the corresponding portions are dissimilar.

By way of illustration, the example method 1100 may be used to compare different biometric templates stored in different records in different systems, such as different medical records systems. For example, the electronic device may compare a first biometric template of a first type, such as one stored in and/or associated with a first medical record in a first medical system, with a second biometric template of a second type, such as one stored in a second medical record in a second medical system. As the first biometric template is of the first type and the second biometric template is of the second type, the electronic device may identify a canonical template structure to which the first and second biometric templates both adhere and use the canonical template structure to compare the first and second biometric templates. Based on the comparison, the electronic device may determine whether or not the records are for the same person by comparing the first and second biometric templates. If the electronic device determines the records are for the same person, the electronic device may link the records, copy information from one record to the other, and so on. Various configurations are possible and contemplated without departing from the scope of the present disclosure.

In some situations, comparison of the first biometric template to the second biometric template using the canonical template structure may not be 100% sufficient for matching. In some cases, additional steps and/or translations may be performed before matching is possible. For example, the first biometric template may be generated at a lower resolution than the second biometric template, which may affect the relationships that are determined among features and how these are represented. In such an example, comparison of the first biometric template to the second biometric template using the canonical template structure may be capable of comparing corresponding portions, but additional translation steps may be performed to account for the differences in how the relationships were determined among features and how these were represented before matching can be adequately certain.

In various examples, this example method 1100 may be implemented as a group of interrelated software modules or components that perform various functions discussed herein. These software modules or components may be executed within a cloud network and/or by one or more computing devices, such as the identification system device 101 of FIG. 1, the biometric template translator 901 of FIG. 9 (and/or the identification system device 1301 of FIG. 13 discussed below).

Although the example method 1100 is illustrated and described as including particular operations performed in a particular order, it is understood that this is an example. In various implementations, various orders of the same, similar, and/or different operations may be performed without departing from the scope of the present disclosure.

For example, the example method 1100 is illustrated and described as accessing both the first biometric template and the second biometric template. However, it is understood that this is an example. In some implementations, the electronic device may generate one or more of the first biometric template or the second biometric template from a biometric instead of accessing such from another source. Various configurations are possible and contemplated without departing from the scope of the present disclosure.

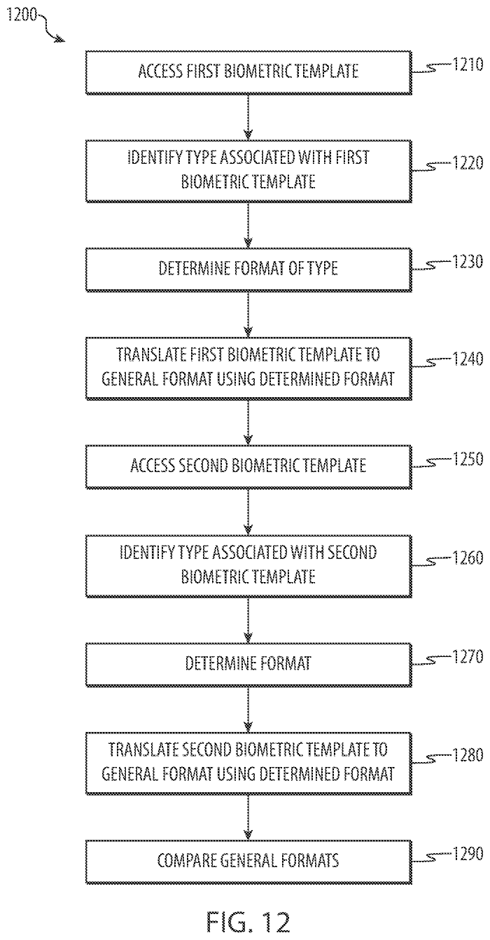

FIG. 12 is a flow chart illustrating a second example method 1200 for biometric template translation. This method 1200 may be performed by the systems 100, 900 of FIG. 1 or 9 (and/or the system 1300 of FIG. 13 discussed below).

At operation 1210, an electronic device (such as the identification system device 101 of FIG. 1, the biometric template translator 901 of FIG. 9, and/or the identification system device 1301 of FIG. 13 discussed below) may access a first biometric template. At operation 1220, the electronic device may identify a type associated with the first biometric template. For example, the first biometric template may be stored in a data structure that identifies the type and/or a matching engine to use with the first biometric template from which the type may be identified. By way of another example, the electronic device may analyze the format of the first biometric template to identify the type. In yet another example, a type indicator may be associated with the first biometric template that identifies the type. Various configurations are possible and contemplated without departing from the scope of the present disclosure.

At operation 1230, the electronic device may determine the format of the type. In other words, the electronic device may determine how all biometric templates of that type are formatted. For example, different types of biometric templates may be formatted differently. The electronic device may access information for different types that specify the formats associated with the respective type. For example, the format may involve how images and/or other data are cropped, features and/or relationships among features are expressed mathematically, what data is represented in what portions, how data may be hashed and/or compressed, and so on.

At operation 1240, the electronic device may translate the first biometric template to a general format using the determined format. The general format may be a standard for biometric templates. The general format may be a “universal” format to which different formatted biometric templates may be converted in order to compare them.

At operation 1250, the electronic device may access a second biometric template. The second biometric template may be of a different type than the first biometric template. At operation 1260, the electronic device may identify the type associated with the second biometric template. At operation 1270, the electronic device may determine a format of the type associated with the second biometric template. At operation 1280, the electronic device may translate the second biometric template to the general format using the determined format.

At operation 1290, the electronic device may compare the general format versions of the first and second biometric templates. The electronic device may compare the general format versions of the first and second biometric templates to determine whether or not they represent the same biometric and/or are from the same person.

By way of illustration, the electronic device may access a first biometric template of a first type via a first external system and a second biometric template of a second type via a second external system, such as by receiving the first biometric template from the first external system and/or the second biometric template from the second external system. The first biometric template may be associated with a first record stored by the first external system and the second biometric template may be associated with a second record stored by the second external system. The electronic device may determine how biometric templates of the first type are formatted, use that determination of how biometric templates of the first type are formatted to translate the first biometric template to a general format, determine how biometric templates of the second type are formatted, use that determination of how biometric templates of the second type are formatted to translate the second biometric template to a general format, and compare the general format versions of the first and second biometric templates.

In various examples, this example method 1200 may be implemented as a group of interrelated software modules or components that perform various functions discussed herein. These software modules or components may be executed within a cloud network and/or by one or more computing devices, such as the identification system device 101 of FIG. 1, the biometric template translator 901 of FIG. 9 (and/or the identification system device 1301 of FIG. 13 discussed below).

Although the example method 1200 is illustrated and described as including particular operations performed in a particular order, it is understood that this is an example. In various implementations, various orders of the same, similar, and/or different operations may be performed without departing from the scope of the present disclosure.

For example, the example method 1200 illustrates and describes converting both the first and second biometric templates to the general format. However, it is understood that this is an example. In some examples, one or more of the first biometric template and/or the second biometric template may already be in the general format and/or a format that is comparable to the general format (such as one that adheres to a canonical biometric structure like discussed above with respect to FIG. 11). In such an example, translation of that biometric template to the general format may be omitted. Various configurations are possible and contemplated without departing from the scope of the present disclosure.

FIG. 13 depicts a second example system 1300 for biometric template translation. The system includes one or more identification system devices 1301 that are operable to communicate with one or more first systems 1302 and/or second systems 1303 via one or more networks 1304.

The identification system device 1301 may be operable to communicate with the first system 1302 and/or the second system 1303 to translate biometric templates of different types and/or perform other functions. For example, the identification system device 1301 may be operable to perform identifications using biometric templates received from the first system 1302 and/or the second system 1303 via the network 1304. By way of another example, the identification system device 1301 may be operable to compare biometric data stored by the first system 1302 and the second system 1303, which may involve translating between different types of biometric templates used to generate the respective biometric data. In yet another example, the identification system device 1301 may identify a person using a biometric template received from the first system 1302 or the second system 1303 and translate the biometric template in one or more various ways to compare that to and/or otherwise evaluate biometric data stored by the other of the first system 1302 and/or the second system 1303.

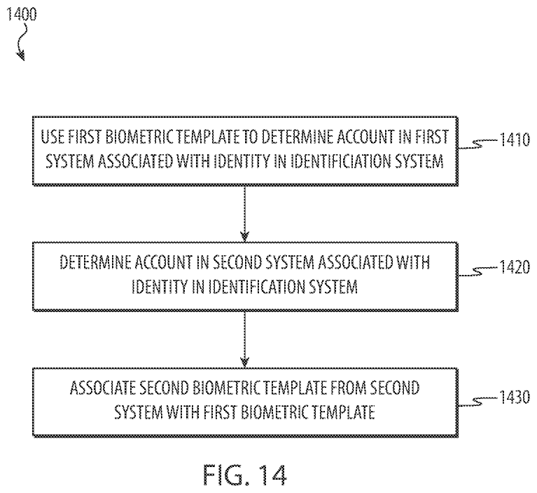

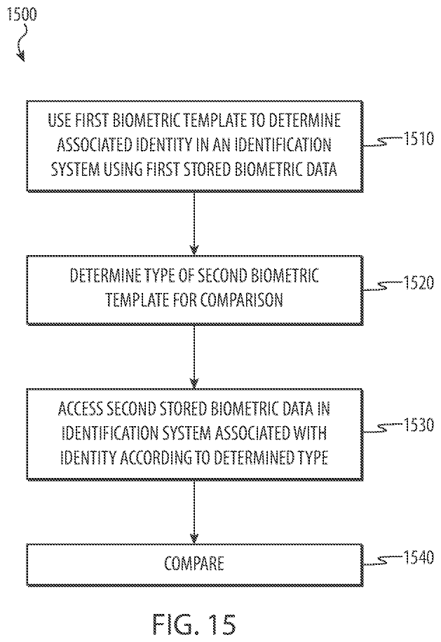

The identification system device 1301 may translate between biometric templates associated with the first system 1302 and/or the second system 1303. The identification system device 1301 may perform one or more methods for translating biometric templates, such as the method 1100 of FIG. 11, the method 1200 of FIG. 12, the method 1400 of FIG. 14 detailed below, the method 1500 of FIG. 15 detailed below, and so on.

In one example, identification system device 1301 may identify a canonical template structure to which the first and second biometric templates adhere and compare corresponding portions of the first and second biometric templates. In another example, identification system device 1301 may identify types and associated formats of the first and second biometric templates in order to translate one or more to a general format that can then be compared. In still other examples, the identification system device 1301 may use the first biometric template to determine an identity associated with first biometric data and retrieve second biometric data of a same type as the second biometric template (such as where the first and second biometric data are both stored in association with the identity). In still other examples, the identification system device 1301 may use the first biometric template to determine an identity associated with first biometric data and verify that an account in one or more of the first system 1302 and/or the second system 1303 having a record storing the second biometric template is also associated with the identity, thus allowing translation between the first and second biometric templates through mutual association with the same identity.

The identification system device 1301 may be any kind of electronic device and/or cloud and/or other computing arrangement and may include one more processing units 1321 and/or other processors or controllers, non-transitory storage media 1322, communication units 1323, and/or other components. The processing units 1321 may execute one or more sets of instructions stored in the non-transitory storage medium 1322 to perform various functions, such as storing biometric data for people and associated identity information (such as one or more names, addresses, telephone numbers, financial data, financial account numbers, verified ages, insurance identifiers, payment account identifiers, and so on), receiving one or more digital representations of biometrics, matching one or more received digital representations of biometrics to stored biometric data, retrieving identity information associated with stored biometric data matching one or more received digital representations of biometrics, providing retrieved identity information, communicating with the first system 1302 and/or the second system 1303 via the network 1304 using the communication unit 1323, translating between one or more types of biometric templates, and so on.

Likewise, the first system 1302 and/or the second system 1303 may be any kind of electronic devices. Examples of such devices include, but are not limited to, one or more desktop computing devices, laptop computing devices, mobile computing devices, wearable devices, tablet computing devices, mobile telephones, smart phones, printers, displays, kiosks, vehicles, kitchen appliances, entertainment system devices, digital media players, and so on. The devices may include one or more processing units and/or other processors and/or controllers, one or more non-transitory storage media (which may take the form of, but is not limited to, a magnetic storage medium; optical storage medium; magneto-optical storage medium; read only memory; random access memory; erasable programmable memory; flash memory; and so on), one or more input and/or output devices (such as one or more keyboards, computer mice, touch screens, touch pads, track pads, microphones, speakers, displays, buttons, dials, switches, printers, and so on), one or more communication units, one or more biometric reader devices (such as a fingerprint scanner, a blood vessel scanner, a palm-vein scanner, an optical fingerprint scanner, a phosphorescent fingerprint scanner, a still image and/or video camera, a 2D and/or 3D image sensor, a capacitive sensor, a saliva sensor, a deoxyribonucleic acid sensor, a heart rhythm monitor, a microphone, and so on), and/or one or more other components. The processing units may execute one or more sets of instructions stored in the non-transitory storage media to perform various functions, such as using the biometric reader device to obtain one or more digital representations of one or more biometrics (such as one or more hashes and/or other digital representations of one or more fingerprints, blood vessel scans, palm-vein scans, voiceprints, facial images, retina images, iris images, deoxyribonucleic acid sequences, heart rhythms, gaits, and so on) for a person, communicating with the identification system device 1301 via the network 1304 using the communication unit 1323, providing one or more obtained digital representations of biometrics, and so on.

FIG. 14 is a flow chart illustrating a third example method 1400 for biometric template translation. This method 1400 may be performed by the systems 100, 900, 1300 of FIG. 1, 9, or 13.

At operation 1410, an electronic device (such as identification system device 101 of FIG. 1, the biometric template translator 901 of FIG. 9, and/or the identification system device 1301 of FIG. 13) may use a first biometric template to determine an account in a first system associated with an identity in an identification system. For example, the electronic device may receive the first biometric template as part of a person interacting with the first system, such as where the person provides a biometric to the first system in order to identify himself for the purpose of checking in for a medical appointment. The first system may generate or obtain the first biometric template, transmit the first biometric template to the identification system, receive information regarding an identity associated with the first biometric template in the identification system, and determine an account in the first system associated with the identity.

At operation 1420, the electronic device may determine an account in a second system associated with the identity in the identification system. For example, the identification system may store information regarding various accounts that are associated with the identity, such as the account in the first system and the second system. Upon determining the identity using the first biometric template, the identification system may determine that the identity is associated with the accounts in both the first and the second systems.

The second system may have a second biometric template associated with the account. The second biometric template may be of a different type or format than the first biometric template. As the electronic device has determined that the identity is associated with the account in the first system, the first biometric template, and the account in the second system, the electronic device may determine that this is sufficient to mutually associate the first and second biometric templates with the same identity. As such, the mutual association of the first and second biometric templates with the identity can be effectively used through the identity system to translate between the first and second biometric templates. Thus, at operation 1430, the electronic device may associate the second biometric template with the first biometric template. Association may involve copying the second biometric template to the identification system and/or the first system, treating the first and second biometric templates as equivalent for determining the identity (and thus “translated”), and so on.

In various examples, this example method 1400 may be implemented as a group of interrelated software modules or components that perform various functions discussed herein. These software modules or components may be executed within a cloud network and/or by one or more computing devices, such as the identification system device 101 of FIG. 1, the biometric template translator 901 of FIG. 9, and/or the identification system device 1301 of FIG. 13.

Although the example method 1400 is illustrated and described as including particular operations performed in a particular order, it is understood that this is an example. In various implementations, various orders of the same, similar, and/or different operations may be performed without departing from the scope of the present disclosure.

For example, the example method 1400 is illustrated and described as using the first biometric template to identify an account in the first system associated with the identity. However, in various examples, a first biometric template may be used to determine the identity for the first system without involving any kind of account in the first system. In such an example, the first biometric template may still be associated with a second biometric template from a second system identified as associated with the identity. Various configurations are possible and contemplated without departing from the scope of the present disclosure.

FIG. 15 is a flow chart illustrating a fourth example method 1500 for biometric template translation. This method 1500 may be performed by the systems 100, 900, 1300 of FIG. 1, 9, or 13.

At operation 1510, an electronic device (such as the identification system device 101 of FIG. 1, the biometric template translator 901 of FIG. 9, and/or the identification system device 1301 of FIG. 13) may use a first biometric template to determine an associated identity in an identification system using first stored biometric data. For example, the identification system may associate the first stored biometric data with the identity and may match the first biometric template to the first stored biometric data to determine the identity and/or control access to information associated with the identity.

At operation 1520, the electronic device may determine a type of a second biometric template to be compared. The second biometric template may be for comparing to the first stored biometric data and/or the first biometric template. The first biometric template and/or the first biometric data may be of a different type than the second biometric template.

At operation 1530, the electronic device may access second stored biometric data in the identification system associated with the identity according to the determined type. The identification system may store biometric data of a number of different types (e.g., different template types) associated with the identity. As such, upon determining the identity using the first biometric template and determining a type of biometric data to use for comparison with a second biometric template, the identification system can serve as a translator by retrieving stored second biometric data of a corresponding type to the second biometric template.

At operation 1540, the electronic device can compare the second biometric template with the first biometric template and/or the first stored biometric data by comparing the second stored biometric data with the second biometric template. As the identity is thus associated in the identification system with the first biometric template, the first stored biometric data, and the second stored biometric data, the second stored biometric data can serve as a stand in for the first biometric data and/or the first biometric template.

In various examples, this example method 1500 may be implemented as a group of interrelated software modules or components that perform various functions discussed herein. These software modules or components may be executed within a cloud network and/or by one or more computing devices, such as the identification system device 101 of FIG. 1, the biometric template translator 901 of FIG. 9, and/or the identification system device 1301 of FIG. 13.

Although the example method 1500 is illustrated and described as including particular operations performed in a particular order, it is understood that this is an example. In various implementations, various orders of the same, similar, and/or different operations may be performed without departing from the scope of the present disclosure.

For example, the example methods 1400 and 1500 are illustrated and described as separate processes. However, in some examples, one or more operations may be combined from the example methods 1400 and 1500 without departing from the scope of the present disclosure.

For example, in some implementations, the example method 1500 may include the further operations of associating the first biometric template and/or the second biometric template with a third biometric template stored in a record in an external system when the identity is associated with an account in the external system that corresponds to the record. In such an example, the electronic device may store the third biometric template in association with the identity upon associating the first biometric template and/or the second biometric template with the third biometric template. Various configurations are possible and contemplated without departing from the scope of the present disclosure.

Although the above illustrates and describes a number of embodiments, it is understood that these are examples. In various implementations, various techniques of individual embodiments may be combined without departing from the scope of the present disclosure.

In various implementations, a system for biometric template translation may include at least one non-transitory storage medium that stores instructions and at least one processing unit. The at least one processing unit may execute the instructions to access a first biometric template of a first type and a second biometric template of a second type, identify a canonical template structure to which the first biometric template and the second biometric template adhere, and compare the first biometric template and the second biometric template using the canonical template structure.

In some examples, the at least one processing unit may compare the first biometric template and the second biometric template by using the canonical template structure to identify corresponding portions of the first biometric template and the second biometric template and comparing the corresponding portions. In various such examples, the at least one processing unit may determine that the first biometric template and the second biometric template are from a same person when the corresponding portions match. In some such examples, the at least one processing unit may determine that the first biometric template and the second biometric template are from different people when the corresponding portions are dissimilar.

In various examples, the at least one processing unit may access the first biometric template from a first record in a first system, access the second biometric template from a second record in a second system, and determine whether the first record and the second record are for a same person by comparing the first biometric template and the second biometric template. In some examples, the first biometric template and the second biometric template may both be digital representations of at least one of fingerprints, blood vessel scans, palm-vein scans, voiceprints, facial images, retina images, iris images, deoxyribonucleic acid sequences, heart rhythms, or gaits. In a number of examples, the canonical template structure may detail standard features of biometric templates that adhere to the canonical template structure.

In some implementations, a system for biometric template translation may include at least one non-transitory storage medium that stores instructions and at least one processing unit. The at least one processing unit may execute the instructions to access a first biometric template, identify a first type of the first biometric template, determine a first format associated with the first type, use the first format to translate the first biometric template to a general format version of the first biometric template, and compare the general format version of the first biometric template with a second biometric template.

In various examples, the at least one processing unit may compare the general format version of the first biometric template with a second biometric template by identifying a second type of the second biometric template, determining a second format associated with the second type, using the second format to translate the second biometric template to a general format version of the second biometric template, and comparing the general format version of the first biometric template with the general format version of the second biometric template.

In some examples, the at least one processing unit may access the first biometric template via a first external system and the second biometric template via a second external system. In various such examples, the first biometric template may be associated with a first record stored by the first external system and the second biometric template may be associated with a second record stored by the second external system. In some such examples, the at least one processing unit may receive the first biometric template from the first external system.

In various examples, the at least one processing unit may identify the first type by analyzing the first biometric template. In some examples, the at least one processing unit may identify the first type using a type indicator associated with the first biometric template.

In a number of implementations, a system for biometric template translation may include at least one non-transitory storage medium that stores instructions and at least one processing unit. The at least one processing unit may execute the instructions to determine an identity of a person in an identification system using a first biometric template and first stored biometric data for the person, determine a type associated with a second biometric template, access second stored biometric data that is associated with the identity in the identification system and corresponds to the type, and compare the second biometric template to the second stored biometric data.

In various examples, the at least one processing unit may associate at least one of the first biometric template or the second biometric template with a third biometric template stored in a record in an external system when the identity is associated with an account in the external system that corresponds to the record. In some such examples, the at least one processing unit may store the third biometric template in association with the identity upon associating the at least one of the first biometric template or the second biometric template with the third biometric template.

In a number of examples, the identification system may store identity information for the person and control access to the identity information using the first stored biometric data. In some examples, the identification system may store multiple sets of biometric data for the person that are each associated with different template types. In various examples, the first biometric template and the second biometric template may be of different types.

As described above and illustrated in the accompanying figures, the present disclosure relates to integrating distributed systems. One or more digital representations of biometrics may be used to match and/or dedupe first and second records respectively stored in first and second systems. Deduping may involve removing duplicate information from one or more of the records and/or the records themselves, linking the records, copying information from one record to the other, and so on. In various examples, an identification system may be used to determine an identity for a first system using a digital representation of a biometric, locate a first record in the first system associated with the identity using the digital representation of the biometric, locate a second record in a second system associated with the identity using the digital representation of the biometric, and dedupe the first and second records. In some examples, an identification system may be used to determine an identity, locate a first record in the first system associated with the identity, locate a second record in a second system associated with the identity, and dedupe the first and second records. In a number of examples, an identification system may be used to locate a first record in a first system using identity information, locate a second record in a second system using the identity information, determine that the first record and second record cannot be verified as associated with a same person within a threshold certainty, and determine whether a person associated with the first record is associated with the second record using a digital representation of a biometric for the person.

The present disclosure recognizes that biometric and/or other personal data is owned by the person from whom such biometric and/or other personal data is derived. This data can be used to the benefit of those people. For example, biometric data may be used to conveniently and reliably identify and/or authenticate the identity of people, access securely stored financial and/or other information associated with the biometric data, and so on. This may allow people to avoid repeatedly providing physical identification and/or other information.

The present disclosure further recognizes that the entities who collect, analyze, store, and/or otherwise use such biometric and/or other personal data should comply with well-established privacy policies and/or privacy practices. Particularly, such entities should implement and consistently use privacy policies and practices that are generally recognized as meeting or exceeding industry or governmental requirements for maintaining security and privately maintaining biometric and/or other personal data, including the use of encryption and security methods that meets or exceeds industry or government standards. For example, biometric and/or other personal data should be collected for legitimate and reasonable uses and not shared or sold outside of those legitimate uses. Further, such collection should occur only after receiving the informed consent. Additionally, such entities should take any needed steps for safeguarding and securing access to such biometric and/or other personal data and ensuring that others with access to the biometric and/or other personal data adhere to the same privacy policies and practices. Further, such entities should certify their adherence to widely accepted privacy policies and practices by subjecting themselves to appropriate third party evaluation.

Additionally, the present disclosure recognizes that people may block the use of, storage of, and/or access to biometric and/or other personal data. Entities who typically collect, analyze, store, and/or otherwise use such biometric and/or other personal data should implement and consistently prevent any collection, analysis, storage, and/or other use of any biometric and/or other personal data blocked by the person from whom such biometric and/or other personal data is derived.

In the present disclosure, the methods disclosed may be implemented as sets of instructions or software readable by a device. Further, it is understood that the specific order or hierarchy of steps in the methods disclosed are examples of sample approaches. In other embodiments, the specific order or hierarchy of steps in the method can be rearranged while remaining within the disclosed subject matter. The accompanying method claims present elements of the various steps in a sample order, and are not necessarily meant to be limited to the specific order or hierarchy presented.

The described disclosure may be provided as a computer program product, or software, that may include a non-transitory machine-readable medium having stored thereon instructions, which may be used to program a computer system (or other electronic devices) to perform a process according to the present disclosure. A non-transitory machine-readable medium includes any mechanism for storing information in a form (e.g., software, processing application) readable by a machine (e.g., a computer). The non-transitory machine-readable medium may take the form of, but is not limited to, a magnetic storage medium (e.g., floppy diskette, video cassette, and so on); optical storage medium (e.g., CD-ROM); magneto-optical storage medium; read only memory (ROM); random access memory (RAM); erasable programmable memory (e.g., EPROM and EEPROM); flash memory; and so on.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. A system for integrating distributed systems, comprising:
at least one non-transitory storage medium that stores instructions; and
at least one processing unit that executes the instructions to:
obtain a digital representation of a biometric;
determine an identity for a first system using the digital representation of the biometric;
locate a first record in the first system associated with the identity using the digital representation of the biometric;
locate a second record in a second system using the digital representation of the biometric; and
dedupe the first record and the second record.

2. The system of claim 1, wherein an entity controls the first system and the second system.

3. The system of claim 1, wherein a first entity controls the first system and a second entity controls the second system.

4. The system of claim 1, wherein:
the first record is associated with a first biometric template of a first type;
the second record is associated with a second biometric template of a second type; and
the at least one processing unit performs biometric template translation in order to compare the first biometric template to the second biometric template.

5. The system of claim 1, wherein the at least one processing unit provides identity information obtained using the identity to the first system.

6. The system of claim 1, wherein the at least one processing unit copies information from the second record to the first record as part of deduping the first record and the second record.

7. The system of claim 1, wherein the at least one processing unit removes information from the first record as part of deduping the first record and the second record.

8. A system for integrating distributed systems, comprising:
at least one non-transitory storage medium that stores instructions; and
at least one processing unit that executes the instructions to:
obtain a digital representation of a biometric;
determine an identity using the digital representation of the biometric;
locate a first record in a first system associated with the identity;
locate a second record in a second system associated with the identity; and
dedupe the first record and the second record.

9. The system of claim 8, wherein the at least one processing unit:
obtains identity information using the identity; and
locates the first record using the identity information.

10. The system of claim 9, wherein the at least one processing unit locates the second record using the identity information.

11. The system of claim 8, wherein the at least one processing unit:
locates the first record by creating a new record; and
dedupes the first record and the second record by copying information from the second record to the new record.

12. The system of claim 8, wherein the first record and the second record are medical records.

13. The system of claim 8, wherein the at least one processing unit obtains permission of a person associated with the identity before exchanging information between the first record and the second record.

14. A system for integrating distributed systems, comprising:
at least one non-transitory storage medium that stores instructions; and
at least one processing unit that executes the instructions to:
use identity information to locate a first record in a first system;
use identity information to locate a second record in a second system;
determine that the first record and the second record cannot be verified as associated with a same person within a threshold certainty;
obtain a digital representation of a biometric for a person associated with the first record; and
use the digital representation of the biometric to determine whether the person is associated with the second record.

15. The system of claim 14, wherein the at least one processing unit dedupes the first record and the second record upon determining that the person is associated with the second record.

16. The system of claim 14, wherein the at least one processing unit links the first record and the second record upon determining that the person is associated with the second record.

17. The system of claim 14, wherein the at least one processing unit requests the person for the digital representation of the biometric.

18. The system of claim 14, wherein the identity information comprises at least one of a name, an address, a birth date, or an identification number.

19. The system of claim 14, wherein:
the digital representation of the biometric is a first biometric template of a first type; and
the second record is associated with a second biometric template of a second type.

20. The system of claim 19, wherein the at least one processing unit translates the first biometric template in order to compare the first biometric template to the second biometric template.
